(19) United States
(12) Patent Application Publication
(10) Pub. No.: US 2021/0068652 A1
(43) Pub. Date: Mar. 11, 2021

(54) Glint-Based Gaze Tracking Using Directional Light Sources

(71) Applicant: Apple Inc., Cupertino, CA (US)

(72) Inventor: Walter Nistico, Redwood City, CA (US)

(21) Appl. No.: 17/010,071

(22) Filed: Sep. 2, 2020

Related U.S. Application Data
(60) Provisional application No. 62/897,540, filed on Sep. 9, 2019.

(51) Int. Cl. A61B 3/113; G06K 9/00; G02B 27/00

(52) U.S. Cl. CPC A61B 3/113; G02B 27/0093; G06K 9/00604

(57) ABSTRACT

Various implementations determine gaze direction based on a cornea center and (a) a pupil center or (b) an eyeball center. The cornea center is determined using a directional light source to produce one or more glints reflected from the surface of the eye and captured by a sensor. The angle (e.g., direction) of the light from the directional light source may be known, for example, using an encoder that records the orientation of the light source. The known direction of the light source facilitates determining the distance of the glint on the cornea and enables the cornea position to be determined, for example, based on a single glint. The cornea center can be determined (e.g., using an average cornea radius, or a previously measured cornea radius or using information from a second glint). The cornea center and a pupil center or eyeball center may be used to determine gaze direction.

### CROSS-REFERENCE TO RELATED APPLICATIONS

This Application claims the benefit of U.S. Provisional Application Ser. No. 62/897,540 filed Sep. 9, 2019, which is incorporated herein in its entirety.

### TECHNICAL FIELD

The present disclosure generally relates to gaze tracking, and in particular, to systems, methods, and devices for gaze tracking using a light source direction of a light source used to produce one or more glints on the surface of the eye.

### BACKGROUND

Some existing gaze tracking systems use light reflected off of the surface of the eye to estimate gaze directions. Such techniques may estimate the user's gaze direction using multiple glints to identify locations along the user's gaze (e.g., pupil center, eye center, and cornea center) or by identifying the user's eye shape, position, and orientation. Existing techniques may be unable to determine gaze direction from a single glint and may not be as accurate or efficient as desired.

### SUMMARY

Various implementations disclosed herein include devices, systems, and methods that determine a gaze direction based on a cornea center and either (a) a pupil center or (b) an eyeball center. The cornea center is determined using a directional light source (e.g., producing a narrow beam light) to produce one or more glints reflected from the surface of the eye and captured by a sensor. Using a directional light source rather than an omnidirectional light source (e.g., producing diffuse light) can provide various advantages. The direction (e.g., angle) of the light from the directional light source may be known, for example, using an encoder that records the orientation of the light source when the light is produced and a reflection identified. A direction (e.g., angle) of the reflection may be determined based on data from a sensor. Using a pixel-based sensor may provide various advantages. For example, pixel data from a camera or other pixel-based sensor may be interpreted to determine the direction of the reflection. The direction of the light source and the direction of the reflection facilitate determining the distance of the position on the surface of the cornea at which the reflection (e.g., glint) occurred. This may enable the cornea position to be determined, for example, based on a single glint. The cornea center can then be determined (e.g., using an average cornea radius or a previously measured cornea radius or using information from a second glint). The cornea center and a pupil center or eyeball center may then be used to determine gaze direction.

Some implementations involve a method of determining gaze direction at an electronic device having a processor. For example, the processor may execute instructions stored in a non-transitory computer-readable medium to determine or track a gaze direction. The method produces a light beam via a light source. The light beam is moved in multiple directions over time and a reflection from a portion of an eye is received at a sensor when the light beam is produced in a first direction of the multiple directions, e.g., a glint is detected. The light source is a directional light source and thus the direction (e.g., angle) of the light source is variable. In some implementations a scanner is configured to scan the light from the light source over multiple angles (e.g., directions) so that the light reflects off various points on the surface of the eye at different times. In some implementations, a scanner is realized as an electro-mechanical assembly with one or two degrees of rotation and one or two motors capable of changing said angles in response to a control signal, and having one encoder per degree of rotation which measures the current angle. The scanner can be used to directly change the main direction of the illumination cone of the light source (if this is mounted on the scanner), or it can do it indirectly by changing the angle(s) of a mirror towards which the light of the light source is directed. As an example, a scanner can use electric motors or servo-motors or galvanometers or piezoelectric actuators to control two rotational joints; or as another example it can be a MEMS mirror. Alternatively, a scanner can be achieved without using any moving parts; in this case it is possible to use a plurality of narrow-beam light sources organized in a 1D or 2D array; each light source being pointed at a different angle, and having a control logic which turns on/off a specific light source in response to a control signal, for example turning on the light source which is oriented according to the closest match to the target angle(s) set by the control signal.

When a glint is detected, the associated direction or angle of the light source associated with the light that produced the glint may be identified and used to determine the glint location on the cornea surface and ultimately the gaze direction. In this example, some of the reflections, but not all of the reflections, will produce glints useful in determining gaze direction. The directional light source may be a laser, a vertical-cavity surface-emitting laser (VCSEL), a narrow beam light source, a collimated light source, or the like. An encoder may identify a first angle (e.g., direction) of the light source while producing the light beam so that the direction (e.g., angle) can be associated with a respective glint.

The method determines a second direction of the reflection based on data (e.g., pixel data) produced by the sensor. In some implementations, the sensor is an event camera (e.g., a dynamic vision sensor (DVS)) configured to detect changes in intensity at pixels. In some implementations, the sensor is a frame-based (e.g., shutter-based) camera configured to capture intensity values at pixels.

The method determines a cornea center of the eye based on the first direction of the light beam and the second direction of the reflection. For example, this may involve determining a position of a cornea surface point (e.g., in a 3D space) by triangulating using a first angle (e.g., direction of the light source) and a second angle (e.g., direction of the light from the glint to the sensor). Such a determination may involve determining a depth/distance away of the glint and thus determining a depth/distance away of a point on the cornea surface. Given the position of such a point on the cornea surface, the cornea center may be determined. In some implementations, the cornea center is determined using an average cornea radius. In other implementations, the cornea center is determined using a previously measured cornea radius for that eye, for example through a calibration procedure. In other implementations, the cornea center is determined based on a second glint produced by a second narrow beam light source. In some implementations, a 3D reconstruction of the cornea is determined based on the cornea surface point and other information regarding cornea shape of the user or average users. In some implementations, the cornea center is determined using a single glint.

The method also determines a pupil center of the eye or an eyeball center of the eye, or both. In some implementations, the pupil center (e.g., center of the iris) is determined based on ambient light reflected off of the pupil/iris that is detected at the same or a different sensor. In some implementations, the pupil center (e.g., center of the iris) is determined based on reflected light from another diffuse light source. In some implementations, the eyeball center is determined by fitting a sphere based on cornea points detected over time and based on an assumption that the eyeball will have moved little or not at all relative to a previously determined eyeball position (e.g., at a prior instant in time).

The method determines the gaze direction by determining a direction from the pupil center through the cornea center or a direction from the eyeball center through the cornea center. In some implementations, the gaze direction is determined based on the cornea center, the pupil center, and the cornea center.

In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein. In accordance with some implementations, a non-transitory computer readable storage medium has stored therein instructions, which, when executed by one or more processors of a device, cause the device to perform or cause performance of any of the methods described herein. In accordance with some implementations, a device includes: one or more processors, a non-transitory memory, and means for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 is a block diagram of an example operating environment in accordance with some implementations.

FIG. 2 is a block diagram of an example controller in accordance with some implementations.

FIG. 3 is a block diagram of an example head-mounted device (HMD) in accordance with some implementations.

FIG. 4 is a block diagram of an example head-mounted device (HMD) in accordance with some implementations.

FIG. 5 is a flowchart representation of a method of gaze tracking in accordance with some implementations.

FIG. 6 illustrates a functional block diagram illustrating gaze tracking using a diffuse light source.

FIG. 7 illustrates a functional block diagram illustrating multiple possible cornea positions being identified when gaze tracking using a diffuse light source.

FIG. 8 illustrates a functional block diagram illustrating a single possible cornea position being identified when gaze tracking using a variable angle light source (e.g., a scanner) at a known angle in accordance with some implementations.

FIG. 9 illustrates a functional block diagram illustrating another single possible cornea position being identified when gaze tracking using a variable angle light source (e.g., a scanner) at a known angle in accordance with some implementations.

FIG. 10 illustrates a functional block diagram illustrating gaze tracking based on an assumed cornea radius in accordance with some implementations.

FIG. 11 illustrates a functional block diagram illustrating gaze tracking using two variable angle light sources (e.g., a scanners) at a known angles in accordance with some implementations.

FIG. 12 illustrates a functional block diagram illustrating gaze tracking based on eyeball center in accordance with some implementations.

FIG. 13 illustrates a functional block diagram illustrating gaze tracking using two variable angle light sources (e.g., a scanners) at a known angles in accordance with some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects and/or variants do not include all of the specific details described herein. Moreover, well-known systems, methods, components, devices and circuits have not been described in exhaustive detail so as not to obscure more pertinent aspects of the example implementations described herein.

In various implementations, gaze tracking is used to enable user interaction, provide foveated rendering, or reduce geometric distortion. A gaze tracking system includes a sensor and a processor that performs gaze tracking on data received from the sensor regarding light from a light source reflected off the eye of a user. In various implementations, the sensor includes an event camera with a plurality of light sensors at a plurality of respective locations that, in response to a particular light sensor detecting a change in intensity of light, generates an event message indicating a particular location of the particular light sensor. An event camera may include or be referred to as a dynamic vision sensor (DVS), a silicon retina, an event-based camera, or a frame-less camera. Thus, the event camera generates (and transmits) data regarding changes in light intensity as opposed to a larger amount of data regarding absolute intensity at each light sensor. Further, because data is generated when intensity changes, in various implementations, the light source is configured to emit light with modulating intensity.

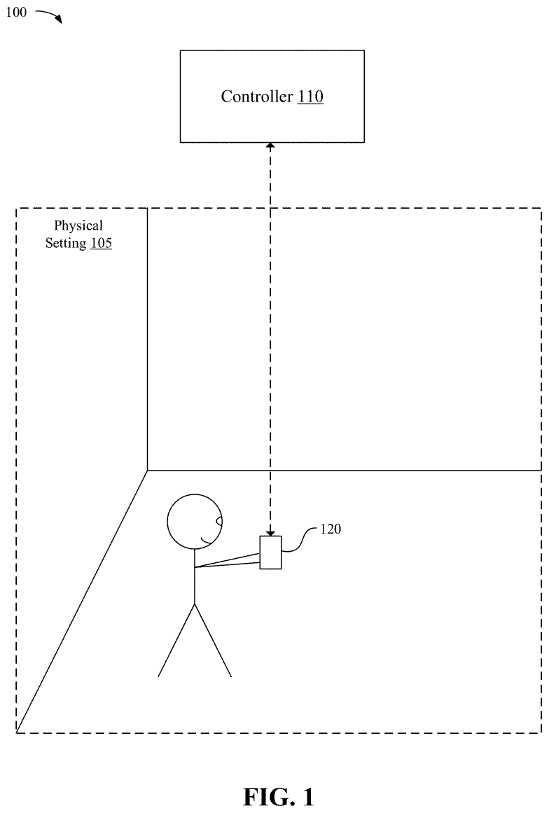

FIG. 1 is a block diagram of an example operating environment 100 in accordance with some implementations. While pertinent features are shown, those of ordinary skill in the art will appreciate from the present disclosure that various other features have not been illustrated for the sake of brevity and so as not to obscure more pertinent aspects of the example implementations disclosed herein. To that end, as a non-limiting example, the operating environment 100 includes a controller 110 and a device 120.

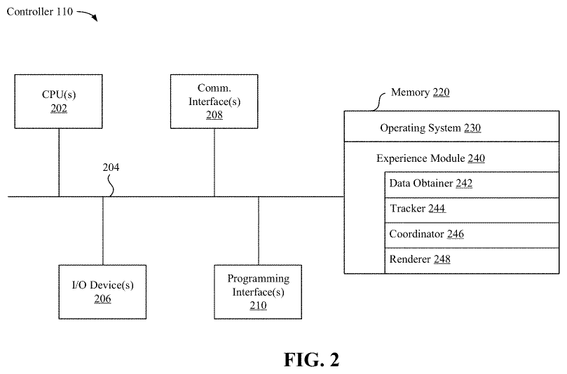

In some implementations, the controller 110 is configured to manage and coordinate a experience for the user. In some implementations, the controller 110 includes a suitable combination of software, firmware, and/or hardware. The controller 110 is described in greater detail below with respect to FIG. 2. In some implementations, the controller 110 is a computing device that is local or remote relative to the physical setting 105. In one example, the controller 110 is a local server located within the physical setting 105. In another example, the controller 110 is a remote server located outside of the physical setting 105 (e.g., a cloud server, central server, etc.). In some implementations, the controller 110 is communicatively coupled with the device 120 via one or more wired or wireless communication channels 144 (e.g., BLUETOOTH, IEEE 802.11x, IEEE 802.16x, IEEE 802.3x, etc.).

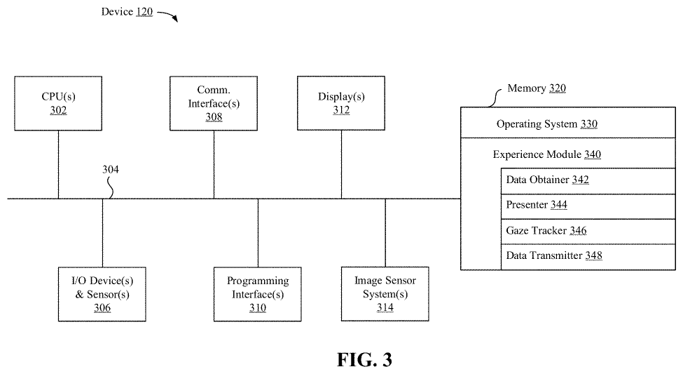

In some implementations, the device 120 is configured to present an environment to the user. In some implementations, the device 120 includes a suitable combination of software, firmware, and/or hardware. The device 120 is described in greater detail below with respect to FIG. 3. In some implementations, the functionalities of the controller 110 are provided by and/or combined with the device 120.

According to some implementations, the device 120 presents a simulated reality (SR) setting (e.g., an augmented reality/virtual reality (AR/VR) setting) to the user while the user is virtually and/or physically present within the physical setting 105. In some implementations, while presenting an experience, the device 120 is configured to present content and to enable optical see-through of the physical setting 105. In some implementations, while presenting a setting, the device 120 is configured to present VR content and to enable video pass-through of the physical setting 105.

In some implementations, the user wears the device 120 on his/her head. As such, the device 120 may include one or more displays provided to display content. For example, the device 120 may enclose the field-of-view of the user. In some implementations, the device 120 is a handheld electronic device (e.g., a smartphone or a tablet) configured to present content to the user. In some implementations, the device 120 is replaced with a chamber, enclosure, or room configured to present content in which the user does not wear or hold the device 120.

FIG. 2 is a block diagram of an example of the controller 110 in accordance with some implementations. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the controller 110 includes one or more processing units 202 (e.g., microprocessors, application-specific integrated-circuits (ASICs), field-programmable gate arrays (FPGAs), graphics processing units (GPUs), central processing units (CPUs), processing cores, and/or the like), one or more input/output (I/O) devices 206, one or more communication interfaces 208 (e.g., universal serial bus (USB), FIREWIRE, THUNDERBOLT, IEEE 802.3x, IEEE 802.11x, IEEE 802.16x, global system for mobile communications (GSM), code division multiple access (CDMA), time division multiple access (TDMA), global positioning system (GPS), infrared (IR), BLUETOOTH, ZIGBEE, and/or the like type interface), one or more programming (e.g., I/O) interfaces 210, a memory 220, and one or more communication buses 204 for interconnecting these and various other components.

In some implementations, the one or more communication buses 204 include circuitry that interconnects and controls communications between system components. In some implementations, the one or more I/O devices 206 include at least one of a keyboard, a mouse, a touchpad, a joystick, one or more microphones, one or more speakers, one or more image sensors, one or more displays, and/or the like.

The memory 220 includes high-speed random-access memory, such as dynamic random-access memory (DRAM), static random-access memory (SRAM), double-data-rate random-access memory (DDR RAM), or other random-access solid-state memory devices. In some implementations, the memory 220 includes non-volatile memory, such as one or more magnetic disk storage devices, optical disk storage devices, flash memory devices, or other non-volatile solid-state storage devices. The memory 220 optionally includes one or more storage devices remotely located from the one or more processing units 202. The memory 220 comprises a non-transitory computer readable storage medium. In some implementations, the memory 220 or the non-transitory computer readable storage medium of the memory 220 stores the following programs, modules and data structures, or a subset thereof including an optional operating system 230 and an experience module 240.

The operating system 230 includes procedures for handling various basic system services and for performing hardware dependent tasks. In some implementations, the presentation module 240 is configured to manage and coordinate one or more experiences for one or more users (e.g., a single experience for one or more users, or multiple experiences for respective groups of one or more users). To that end, in various implementations, the presentation module 240 includes a data obtainer 242, a presenter 244, a gaze tracker 246, and a data transmitter 248.

In some implementations, the data obtainer 242 is configured to obtain data from at least device 120. To that end, in various implementations, the data obtainer 242 includes instructions and/or logic therefor, and heuristics and metadata therefor.

In some implementations, the presenter 244 is configured to present content via the one or more displays 212. To that end, in various implementations, the presenter 244 includes instructions and/or logic therefor, and heuristics and metadata therefor.

In some implementations, the gaze tracker 246 is configured to determine a gaze direction of a user via one or more of the techniques disclosed herein. To that end, in various implementations, the gaze tracker 246 includes instructions and/or logic therefor, configured neural networks, and heuristics and metadata therefor.

In some implementations, the data transmitter 248 is configured to transmit data to at least the device 120. To that end, in various implementations, the data transmitter 248 includes instructions and/or logic therefor, and heuristics and metadata therefor.

Moreover, FIG. 2 is intended more as functional description of the various features which are present in a particular implementation as opposed to a structural schematic of the implementations described herein. As recognized by those of ordinary skill in the art, items shown separately could be combined and some items could be separated. For example, some functional modules shown separately in FIG. 2 could be implemented in a single module and the various functions of single functional blocks could be implemented by one or more functional blocks in various implementations. The actual number of modules and the division of particular functions and how features are allocated among them will vary from one implementation to another and, in some implementations, depends in part on the particular combination of hardware, software, and/or firmware chosen for a particular implementation.

FIG. 3 is a block diagram of an example of the device 120 in accordance with some implementations. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 120 includes one or more processing units 302 (e.g., microprocessors, ASICs, FPGAs, GPUs, CPUs, processing cores, and/or the like), one or more input/output (I/O) devices and sensors 306, one or more communication interfaces 308 (e.g., USB, FIREWIRE, THUNDERBOLT, IEEE 802.3x, IEEE 802.11x, IEEE 802.16x, GSM, CDMA, TDMA, GPS, IR, BLUETOOTH, ZIGBEE, SPI, I2C, and/or the like type interface), one or more programming (e.g., I/O) interfaces 310, one or more AR/VR displays 312, one or more interior and/or exterior facing image sensor systems 314, a memory 320, and one or more communication buses 304 for interconnecting these and various other components.

In some implementations, the one or more communication buses 304 include circuitry that interconnects and controls communications between system components. In some implementations, the one or more I/O devices and sensors 306 include at least one of an inertial measurement unit (IMU), an accelerometer, a magnetometer, a gyroscope, a thermometer, one or more physiological sensors (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), and/or the like.

In some implementations, the one or more displays 312 are configured to present the experience to the user. In some implementations, the one or more displays 312 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QD-LED), micro-electromechanical system (MEMS), and/or the like display types. In some implementations, the one or more displays 312 correspond to diffractive, reflective, polarized, holographic, etc. waveguide displays. For example, the device 120 includes a single display. In another example, the device 120 includes an display for each eye of the user. In some implementations, the one or more displays 312 are capable of presenting SR content.

In some implementations, the one or more image sensor systems 314 are configured to obtain image data that corresponds to at least a portion of the face of the user that includes the eyes of the user. For example, the one or more image sensor systems 314 include one or more RGB cameras (e.g., with a complimentary metal-oxide-semiconductor (CMOS) image sensor or a charge-coupled device (CCD) image sensor), monochrome cameras, IR cameras, event-based cameras, and/or the like. In various implementations, the one or more image sensor systems 314 further include illumination sources that emit light upon the portion of the face of the user, such as a flash or a glint source.

The memory 320 includes high-speed random-access memory, such as DRAM, SRAM, DDR RAM, or other random-access solid-state memory devices. In some implementations, the memory 320 includes non-volatile memory, such as one or more magnetic disk storage devices, optical disk storage devices, flash memory devices, or other non-volatile solid-state storage devices. The memory 320 optionally includes one or more storage devices remotely located from the one or more processing units 302. The memory 320 comprises a non-transitory computer readable storage medium. In some implementations, the memory 320 or the non-transitory computer readable storage medium of the memory 320 stores the following programs, modules and data structures, or a subset thereof including an optional operating system 330, an AR/VR presentation module 340, and a user data store 360.

The operating system 330 includes procedures for handling various basic system services and for performing hardware dependent tasks. In some implementations, the presentation module 340 is configured to present content to the user via the one or more displays 312. To that end, in various implementations, the presentation module 340 includes a data obtainer 342, a presenter 344, a gaze tracker 346, and a data transmitter 348.

In some implementations, the data obtainer 342 is configured to obtain data (e.g., presentation data, interaction data, sensor data, location data, etc.) from at least the controller 110. To that end, in various implementations, the data obtainer 342 includes instructions and/or logic therefor, and heuristics and metadata therefor.

In some implementations, the presenter 344 is configured to present content via the one or more displays 312. To that end, in various implementations, the presenting unit 344 includes instructions and/or logic therefor, and heuristics and metadata therefor.

In some implementations, the gaze tracker 346 is configured to determine a gaze direction of a user via one or more of the techniques disclosed herein. To that end, in various implementations, the gaze tracker 346 includes instructions and/or logic therefor, configured neural networks, and heuristics and metadata therefor.

In some implementations, the data transmitter 348 is configured to transmit data (e.g., presentation data, location data, etc.) to at least the controller 110. To that end, in various implementations, the data transmitter 348 includes instructions and/or logic therefor, and heuristics and metadata therefor.

Although these elements are shown as residing on a single device (e.g., the device 120), it should be understood that in other implementations, any combination of the elements may be located in separate computing devices. Moreover, FIG. 3 is intended more as functional description of the various features which are present in a particular implementation as opposed to a structural schematic of the implementations described herein. As recognized by those of ordinary skill in the art, items shown separately could be combined and some items could be separated. For example, some functional modules shown separately in FIG. 3 could be implemented in a single module and the various functions of single functional blocks could be implemented by one or more functional blocks in various implementations. The actual number of modules and the division of particular functions and how features are allocated among them will vary from one implementation to another and, in some implementations, depends in part on the particular combination of hardware, software, and/or firmware chosen for a particular implementation.

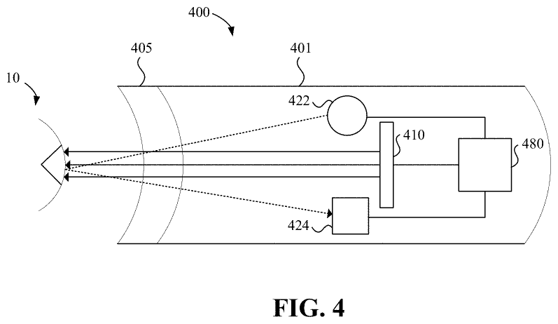

FIG. 4 illustrates a block diagram of a head-mounted device 400 in accordance with some implementations. The head-mounted device 400 includes a housing 401 (or enclosure) that houses various components of the head-mounted device 400. The housing 401 includes (or is coupled to) an eye pad 405 disposed at a proximal (to the user 10) end of the housing 401. In various implementations, the eye pad 405 is a plastic or rubber piece that comfortably and snugly keeps the head-mounted device 400 in the proper position on the face of the user 10 (e.g., surrounding the eye of the user 10).

The housing 401 houses a display 410 that displays an image, emitting light towards onto the eye of a user 10. In various implementations, the display 410 emits the light through an eyepiece (not shown) that refracts the light emitted by the display 410, making the display appear to the user 10 to be at a virtual distance farther than the actual distance from the eye to the display 410. For the user to be able to focus on the display 410, in various implementations, the virtual distance is at least greater than a minimum focal distance of the eye (e.g., 7 cm). Further, in order to provide a better user experience, in various implementations, the virtual distance is greater than 1 meter.

Although FIG. 4 illustrates a head-mounted device 400 including a display 410 and an eye pad 405, in various implementations, the head-mounted device 400 does not include a display 410 or includes an optical see-through display without including an eye pad 405.

The housing 401 also houses a gaze tracking system including one or more light sources 422, camera 424, and a controller 480. The one or more light sources 422 emit light onto the eye of the user 10 that reflects light (e.g., a directional beam) that can be detected by the sensor 424. Based on the reflected glint(s), the controller 480 can determine a gaze direction of the user 10. As another example, the controller 480 can determine a pupil center, a pupil size, or a point of regard. Thus, in various implementations, the light is emitted by the one or more light sources 422, reflects off the eye of the user 10, and is detected by the sensor 424. In various implementations, the light from the eye of the user 10 is reflected off a hot mirror or passed through an eyepiece before reaching the sensor 424.

The display 410 may emit light in a first wavelength range and the one or more light sources 422 may emit light in a second wavelength range. Similarly, the sensor 424 may detects light in the second wavelength range. In various implementations, the first wavelength range is a visible wavelength range (e.g., a wavelength range within the visible spectrum of approximately 400-700 nm) and the second wavelength range is a near-infrared wavelength range (e.g., a wavelength range within the near-infrared spectrum of approximately 700-1400 nm).

In various implementations, gaze tracking (or, in particular, a determined gaze direction) is used to enable user interaction (e.g., the user 10 selects an option on the display 410 by looking at it), provide foveated rendering (e.g., present a higher resolution in an area of the display 410 the user 10 is looking at and a lower resolution elsewhere on the display 410), or reduce geometric distortion (e.g., in 3D rendering of objects on the display 410).

In various implementations, the one or more light sources 422 emit light towards the eye of the user which reflects in the form of one or more glints.

In various implementations, the one or more light sources 422 emit a light beam via to produce a reflection from a portion of an eye. The light source may be a directional light source and thus the angle of the light source is variable. The directional light source may be a laser, a vertical-cavity surface-emitting laser (VCSEL), a narrow beam light source, a collimated light source, or the like.

In various implementations, the one or more light sources 422 include or are coupled to a scanner that is configured to scan the light from the light source over multiple angles (e.g., directions) so that the light reflects off various points on the surface of the eye at different times. The light source may be mounted on a scanner or a mirror mounted on the scanner.

In various implementations, the one or more light sources 422 include or are communicatively coupled to an encoder that is configured to identify or record angles (e.g., directions) of the light source while producing the light beam so that the angle can be associated with respective glints. The light source may be mounted on a scanner/encoder or a mirror mounted on the scanner/encoder.

When a glint, reflected by the eye and detected by the sensor 424, is analyzed, the identity of the glint and the corresponding light source angle (e.g., direction) can be determined.

In various implementations, the one or more light sources 422 include multiple lights sources. Each such light source may be coupled with a scanner and/or an encoder to move light beams from the lights sources over the surface of the eye and record the angles (e.g., directions) of the light sources.

In various implementations, the one or more light sources 422 modulate the intensity of emitted light with different modulation frequencies. For example, in various implementations, a first light source of the one or more light sources 422 is modulated at a first frequency (e.g., 600 Hz) and a second light source of the one or more light sources 422 is modulated at a second frequency (e.g., 500 Hz).

In various implementations, the one or more light sources 422 modulate the intensity of emitted light according to different orthogonal codes, such as those which may be used in CDMA (code-divisional multiplex access) communications. For example, the rows or columns of a Walsh matrix can be used as the orthogonal codes. Accordingly, in various implementations, a first light source of the plurality of light sources is modulated according to a first orthogonal code and a second light source of the plurality of light sources is modulated according to a second orthogonal code.

In various implementations, the one or more light sources 422 modulate the intensity of emitted light between a high intensity value and a low intensity value. Thus, at various times, the intensity of the light emitted by the light source is either the high intensity value or the low intensity value. In various implementation, the low intensity value is zero. Thus, in various implementations, the one or more light sources 422 modulate the intensity of emitted light between an on state (at the high intensity value) and an off state (at the low intensity value). In various implementations, the number of light sources of the plurality of light sources in the on state is constant.

In various implementations, the one or more light sources 422 modulate the intensity of emitted light within an intensity range (e.g., between 10% maximum intensity and 40% maximum intensity). Thus, at various times, the intensity of the light source is either a low intensity value, a high intensity value, or some value in between. In various implementations, the one or more light sources 422 are differentially modulated such that a first light source of the plurality of light sources is modulated within a first intensity range and a second light source of the plurality of light sources is modulated within a second intensity range different than the first intensity range.

In various implementations, the one or more light sources 422 modulate the intensity of emitted light according to user biometrics. For example, if the user is blinking more than normal, has an elevated heart rate, or is registered as a child, the one or more light sources 422 decreases the intensity of the emitted light (or the total intensity of all light emitted by the plurality of light sources) to reduce stress upon the eye. As another example, the one or more light sources 422 modulate the intensity of emitted light based on an eye color of the user, as spectral reflectivity may differ for blue eyes as compared to brown eyes.

In various implementations, the one or more light sources 422 modulate the intensity of emitted light according to a presented user interface (e.g., what is displayed on the display 410). For example, if the display 410 is unusually bright (e.g., a video of an explosion is being displayed), the one or more light sources 422 increase the intensity of the emitted light to compensate for potential interference from the display 410.

In various implementations, the sensor 424 is a frame/shutter-based camera that, at a particular point in time or multiple points in time at a frame rate, generates an image of the eye of the user 10. Each image includes a matrix of pixel values corresponding to pixels of the image which correspond to locations of a matrix of light sensors of the camera.

In various implementations, the camera 424 is an event camera comprising a plurality of light sensors (e.g., a matrix of light sensors) at a plurality of respective locations that, in response to a particular light sensor detecting a change in intensity of light, generates an event message indicating a particular location of the particular light sensor.

An event camera is used in some implementations. An event camera may include a plurality of light sensors respectively coupled to a message generator. In various implementations, the plurality of light sensors are arranged in a matrix of rows and columns and, thus, each of the plurality of light sensors is associated with a row value and a column value.

Each of the plurality of light sensors includes a light sensor. The light sensor includes a photodiode in series with a resistor between a source voltage and a ground voltage. The voltage across the photodiode is proportional to the intensity of light impinging on the light sensor. The light sensor includes a first capacitor in parallel with the photodiode. Accordingly, the voltage across the first capacitor is the same as the voltage across the photodiode (e.g., proportional to the intensity of light detected by the light sensor).

The light sensor includes a switch coupled between the first capacitor and a second capacitor. The second capacitor is coupled between the switch and the ground voltage. Accordingly, when the switch is closed, the voltage across the second capacitor is the same as the voltage across the first capacitor (e.g., proportional to the intensity of light detected by the light sensor). When the switch is open, the voltage across the second capacitor is fixed at the voltage across the second capacitor when the switch was last closed.

The voltage across the first capacitor and the voltage across the second capacitor are fed to a comparator. When the difference between the voltage across the first capacitor and the voltage across the second capacitor is less than a threshold amount, the comparator outputs a ‘0’ voltage. When the voltage across the first capacitor is higher than the voltage across the second capacitor by at least the threshold amount, the comparator outputs a ‘1’ voltage. When the voltage across the first capacitor is less than the voltage across the second capacitor by at least the threshold amount, the comparator outputs a ‘−1’ voltage.

When the comparator outputs a ‘1’ voltage or a ‘−1’ voltage, the switch is closed and the message generator receives this digital signal and generates a pixel event message.

As an example, at a first time, the intensity of light impinging on the light sensor is a first light value. Accordingly, the voltage across the photodiode is a first voltage value. Likewise, the voltage across the first capacitor is the first voltage value. For this example, the voltage across the second capacitor is also the first voltage value. Accordingly, the comparator outputs a ‘0’ voltage, the switch remains closed, and the message generator does nothing.

At a second time, the intensity of light impinging on the light sensor increases to a second light value. Accordingly, the voltage across the photodiode is a second voltage value (higher than the first voltage value). Likewise, the voltage across the first capacitor is the second voltage value. Because the switch is open, the voltage across the second capacitor is still the first voltage value. Assuming that the second voltage value is at least the threshold value greater than the first voltage value, the comparator outputs a ‘1’ voltage, closing the switch, and the message generator generates an event message based on the received digital signal.

With the switch closed by the ‘1’ voltage from the comparator, the voltage across the second capacitor is changed from the first voltage value to the second voltage value. Thus, the comparator outputs a ‘0’ voltage, opening the switch.

At a third time, the intensity of light impinging on the light sensor increases (again) to a third light value. Accordingly, the voltage across the photodiode is a third voltage value (higher than the second voltage value). Likewise, the voltage across the first capacitor is the third voltage value. Because the switch is open, the voltage across the second capacitor is still the second voltage value. Assuming that the third voltage value is at least the threshold value greater than the second voltage value, the comparator outputs a ‘1’ voltage, closing the switch, and the message generator generates an event message based on the received digital signal.

With the switch closed by the ‘1’ voltage from the comparator, the voltage across the second capacitor is changed from the second voltage value to the third voltage value. Thus, the comparator outputs a ‘0’ voltage, opening the switch.

At a fourth time, the intensity of light impinging on the light sensor decreases back to second light value. Accordingly, the voltage across the photodiode is the second voltage value (less than the third voltage value). Likewise, the voltage across the first capacitor is the second voltage value. Because the switch is open, the voltage across the second capacitor is still the third voltage value. Thus, the comparator outputs a ‘−1’ voltage, closing the switch, and the message generator generates an event message based on the received digital signal.

With the switch closed by the ‘−1’ voltage from the comparator, the voltage across the second capacitor is changed from the third voltage value to the second voltage value. Thus, the comparator outputs a ‘0’ voltage, opening the switch.

The message generator receives, at various times, digital signals from each of the plurality of light sensors indicating an increase in the intensity of light (1′ voltage) or a decrease in the intensity of light (‘−1’ voltage). In response to receiving a digital signal from a particular light sensor of the plurality of light sensors, the message generator generates a pixel event message.

In various implementations, each pixel event message indicates, in a location field, the particular location of the particular light sensor. In various implementations, the event message indicates the particular location with a pixel coordinate, such as a row value (e.g., in a row field) and a column value (e.g., in a column field). In various implementations, the event message further indicates, in a polarity field, the polarity of the change in intensity of light. For example, the event message may include a ‘1’ in the polarity field to indicate an increase in the intensity of light and a ‘0’ in the polarity field to indicate a decrease in the intensity of light. In various implementations, the event message further indicates, in a time field, a time the change in intensity in light was detected (e.g., a time the digital signal was received). In various implementations, the event message indicates, in an absolute intensity field (not shown), as an alternative to or in addition to the polarity, a value indicative of the intensity of detected light.

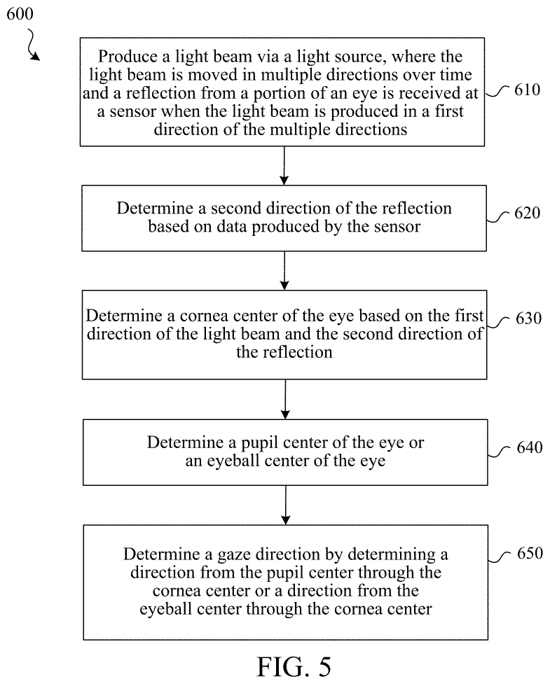

FIG. 5 is a flowchart representation of a method 600 of determining gaze direction in accordance with some implementations. In some implementations, the method 600 is performed by a device (e.g., controller 110 of FIGS. 1 and 2), such as a mobile device, desktop, laptop, or server device. The method 600 can be performed on a device (e.g., device 120 of FIGS. 1 and 3) that has a screen for displaying 2D images and/or a screen for viewing stereoscopic images such as a head-mounted display (HMD). In some implementations, the method 600 is performed by processing logic, including hardware, firmware, software, or a combination thereof. In some implementations, the method 600 is performed by a processor executing code stored in a non-transitory computer-readable medium (e.g., a memory).

At block 610, the method 600 produces a light beam via a light source, where the light beam is moved in multiple directions over time and a reflection from a portion of an eye is received at a sensor when the light beam is produced in a first direction of the multiple directions. For example, a directional light source may produce a light beam at a variety of different angles (e.g., directions). In some implementations a scanner is configured to scan the light from the light source over multiple angles (e.g., directions) so that the light reflects off various points on the surface of the eye at different times. Some reflections, but not all of the reflections, of light beam produced by the directional light source will produce glints useful in determining gaze direction. The directional light source may be a laser, a vertical-cavity surface-emitting laser (VCSEL), a narrow beam light source, a collimated light source, or the like. An encoder may identify a first angle (e.g., direction) of the light source while producing the light beam so that the angle can be associated with a respective glint.

At block 620, the method 600 determines a second direction of the reflection based on data (e.g., pixel data) produced by the sensor. For example, a glint may be detected in an image captured by a frame-based (e.g., shutter-based) camera. In another example, a glint may be detected based on one or more events detected by an event camera. In some implementations, camera data is processed via an algorithm or machine learning model to identify glints or distinguish glints from non-glint-based events. The pixels associated with a reflection (e.g., glint) and/or the sensor's known position or orientation relative to the light source can be used to determine the direction (e.g., angle) the reflection.

At block 630, the method 600 determines a cornea center of the eye based on the first direction of the light beam and the second direction of the reflection. For example, when a glint is detected, the associated angle of the light source associated with the light that produced the glint may be identified and used to determine the glint location on the cornea surface. For example, this may involve determining a position of a cornea surface point (e.g., in a 3D space) by triangulating using the first angle (e.g., direction of the light source) and the second angle (e.g., direction of the light from the glint to the sensor). In some implementations, the method 600 computes a bisector between an incident light ray (measured by a scanner) and a reflected light ray (measured by a sensor). Such determinations may involve determining a depth/distance away of the glint and thus determining a depth/distance away of a point on the cornea surface. Given the position of such a point on the cornea surface, the cornea center may be determined. In some implementations, the cornea center is determined using a known cornea radius, for example an average cornea radius, or a previously measured one. In other implementations, the cornea center is determined based on a second glint produced by a second narrow beam light source. In some implementations, a 3D reconstruction of the cornea is determined based on the cornea surface point and other information regarding cornea shape of the user or average users. In some implementations, the cornea center is determined using a single glint.

At block 640, the method 600 determines a pupil center of the eye or an eyeball center of the eye. In some implementations, the pupil center (e.g., center of the iris) is determined based on ambient light reflected off of the pupil/iris that is detected at the same or a different sensor. In some implementations, the pupil center (e.g., center of the iris) is determined based on reflected light from another diffuse light source. In some implementations, the eyeball center is determined by fitting a sphere based on cornea points detected over time and based on an assumption that the eyeball will have moved little or not at all (e.g., where the eyeball is slow changing or quasi-stationary) relative to a previously determined eyeball position (e.g., at a prior instant in time).

At block 650, the method 600 determines the gaze direction by determining a direction from the pupil center through the cornea center or a direction from the eyeball center through the cornea center. In some implementations, the gaze direction is determined based on the cornea center, the pupil center, and the eyeball center.

The method 600, in some implementations, uses a coherent or very narrow beam light source and a scanner/encoder mechanism instead of an omnidirectional light source to produce glints only under a narrow angle that is measured by the encoder. Measuring an incident illumination ray through the encoder allows the cornea's 3D position to be determined with a single glint. Using such techniques may reduce the number of light sources that may otherwise be required to produce glints and/or to simplify the optomechanical design and integration of the gaze tracking components, e.g., in an HMD. Using narrow beam illumination may reduce the power consumption that may otherwise be required. Using a scanner/encoder allows measuring glint depth with a single camera which, among other things, allows 3D reconstruction of the cornea surface for increased gaze accuracy and determination of the cornea center without needing to know the cornea radius.

The method 600, in some implementations, detects the cornea center based on one or more glints identified using an event camera. Using an event camera to identify glints may provide advantages over techniques that rely solely on shutter-based (e.g., frame-based) camera data. Event cameras may capture data at a very high sample rate and thus allow identification of glints at a faster rate than using a shutter-based camera. In some implementations, images (e.g., the intensity reconstruction images) are created that can emulate data from an extremely fast shutter-based camera without the high energy and data requirements of such a camera. An event camera produces relatively sparse data since it does not collect/send an entire frame for every event. However, the sparse data can be accumulated over time to provide dense input images that may be used as inputs in the gaze direction determinations. The result may be faster gaze tracking enabled using less data and computing resources. Furthermore, a fast readout camera such as the event camera may allow lock-on tracking of the cornea position as a feedback loop between the glint position on the sensor and the angle(s) measured by the scanner/encoder; this may prevent having to repeat the scanning pattern of the beam to find the location of the eye each time the eye moves significantly.

In some implementations, a user's gaze is tracked based on additional information. For example, a correspondence between a selection of a UI item displayed on a screen of a HMD and a gaze direction can be determined when the user selects the UI item based on the assumption that the user is looking at the UI item as he or she selects it. Based on the location of the UI element on the display, the location of the display relative to the user, and the current pupil location, the gaze direction associated with the direction from the eye to the UI element can be determined. Such information can be used to adjust or calibrate gaze tracking performed using the method 600. For example, it may be used to adjust an assumed cornea radius.

Gaze direction can be used for numerous purposes. In one example, the gaze direction that is determined or updated is used to identify an item displayed on a display, e.g., to identify what button, image, text, or other user interface item a user is looking at. In another example, the gaze characteristic that is determined or updated is used to display a movement of a graphical indicator (e.g., a cursor or other user controlled icon) on a display. In another example, the gaze characteristic that is determined or updated is used to select an item (e.g., via a cursor selection command) displayed on a display. For example, a particular gaze movement pattern can be recognized and interpreted as a particular command.

In some implementations, the gaze tracking is performed on two eyes of a same individual concurrently. In implementations in which images of both eyes are captured or derived, the system may determine or produce output useful in determining a convergence point of gaze directions from the two eyes. The system could additionally or alternatively be configured to account for extraordinary circumstances such as optical axes that do not align.

In some implementations, post-processing of gaze direction is employed. Noise in the tracked gaze direction can be reduced using filtering and prediction methods, for example, using a Kalman filter. These methods can also be used for interpolation/extrapolation of the gaze direction over time. For example, the methods can be used if the state of the gaze direction is required at a timestamp different from the recorded states.

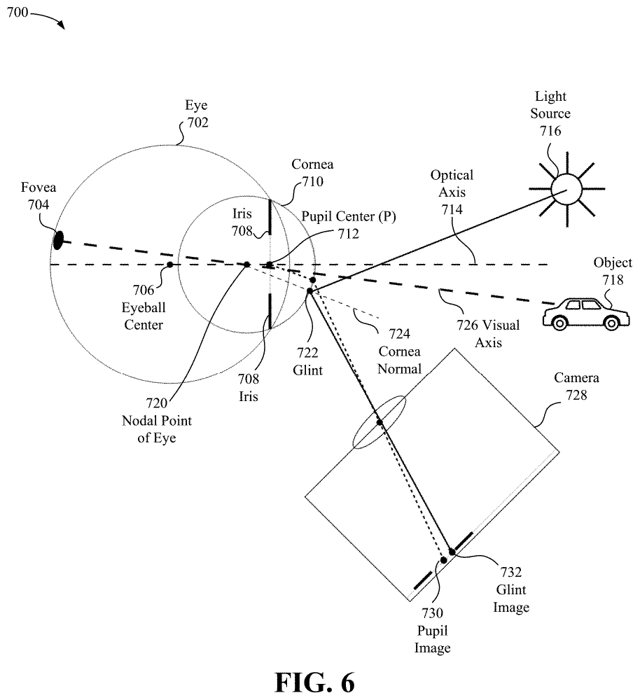

FIG. 6 illustrates a functional block diagram illustrating gaze tracking using a diffuse light source. In this example, eye 702 is gazing in object 718. Light from the object 718 travels through the eye 702 and forms an image on the fovea 704 of the eye 702. In this example, the cornea 710, iris 708, eyeball center 706, nodal point of eye 720, pupil center 712, visual axis 726, and optical axis 714 are depicted. A gaze tracking system including light source 716 and camera 728 is used to track the gaze direction. The light source produces omnidirectional light that produces glint 722 by reflecting off the surface of the cornea 710. The camera 728 captures an image that includes a pupil image 730 and a glint image 732. Using a gaze tracking system as illustrated in FIG. 6 requires at least two light sources, a camera, and a known or assumed cornea radius to measure the cornea 710 position. If cornea radius is not assumed to be constant, at least two cameras are need.

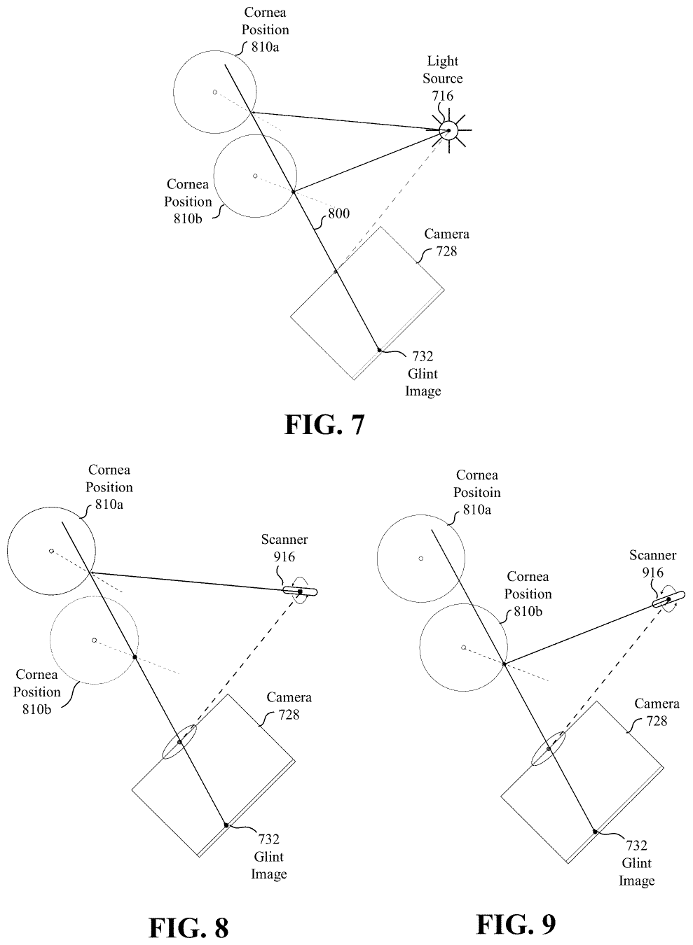

These issues are illustrated in FIG. 7. FIG. 7 illustrates that multiple possible cornea positions 810a, 810b may be identified when gaze tracking using a diffuse light source. Based on the single glint, the cornea's position may be anywhere along path 800.

In contrast, FIGS. 8 and 9 use a directional lights source with scanner 916 and can identify the cornea's actual position based on the single glint. The appropriate cornea position is identified based on the angle of the scanner 916. In FIG. 8, the angle of the scanner 916 identifies cornea position 810a as the appropriate cornea position and, in FIG. 9, the angle of the scanner 916 identifies the cornea position 810b as the actual cornea position.

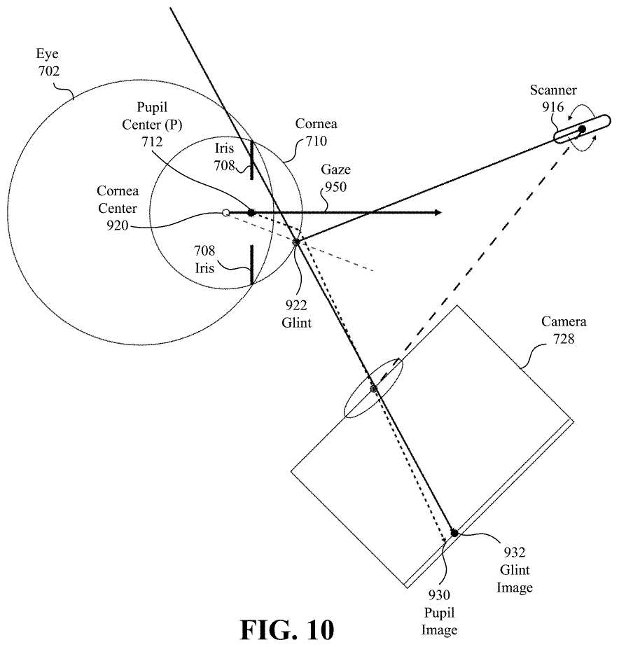

In some implementations, a cornea radius (e.g., 7.8 mm average of population) is assumed and used to determine the center of the cornea curvature. FIG. 10 illustrates gaze tracking based on an assumed cornea radius in accordance with some implementations. In this example, glint 922 produced by a light beam from a light source directed by scanner 916 forms a glint image 932 at the camera 728. The glint 922 and angle of the scanner 916 are used to determine the glint 922 location. The cornea center 920 is then determined using the glint 922 location, lying on the bisector vector between the glint vector and the incident beam angle (measured by the scanner) and at a distance from the 3D glint location equal to the known cornea (assumed average or previously measured) radius. The pupil center 712 is also determined, for example, using image processing on the image data obtained by the camera 728. For example, based on determining the center of the iris 708. The gaze direction 950 in these examples is determined as the vector from the cornea center 920 through the pupil center 712.

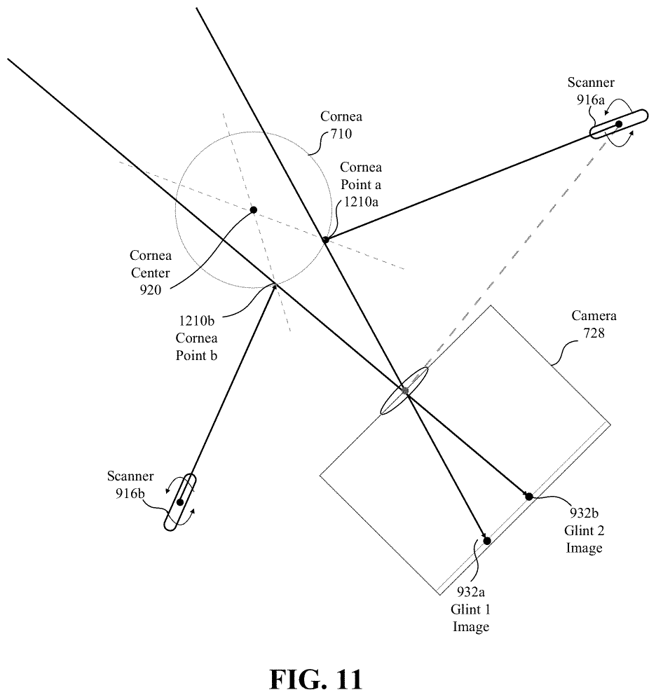

In some implementations, multiple lights sources and/or scanners are used to determine the center of the cornea. FIG. 11 illustrates a functional block diagram illustrating gaze tracking using two variable angle light sources (e.g., a scanners 916a and 916b) at a known angles. In this example, a first glint produced by a light beam from a light source directed by scanner 916a forms a glint image 932a at the camera 728. The glint and angle of the scanner 916a are used to determine cornea point a 1210a. The second glint produced by a light beam from a light source directed by scanner 916b forms a glint image 932b at the camera 728. The glint and angle of the scanner 916b are used to determine cornea point a 1210b. Cornea points 916a, 916b can then be used to determine the cornea center 920. The cornea center 920 may be determined with high accuracy and without needing to assume a standard cornea radius, e.g., by finding the intersection or closest point of the two bisector normal.

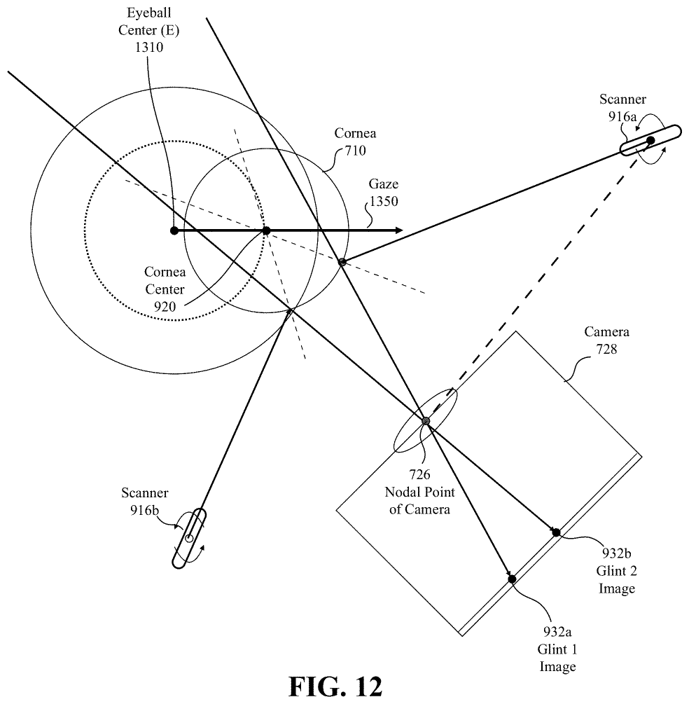

In some implementations, gaze tracking may be performed based on an assumption (or measuring) that the eyeball center does not move so that the gaze direction may be determined without detecting the pupil. FIG. 12 illustrates gaze tracking based on eyeball center in accordance with some implementations. In this example, the eyeball center 1310 is determined by fitting a sphere on cornea center movements. The gaze direction 1350 may then be determined by determining the vector from the eyeball center 1310 through the cornea center 920.

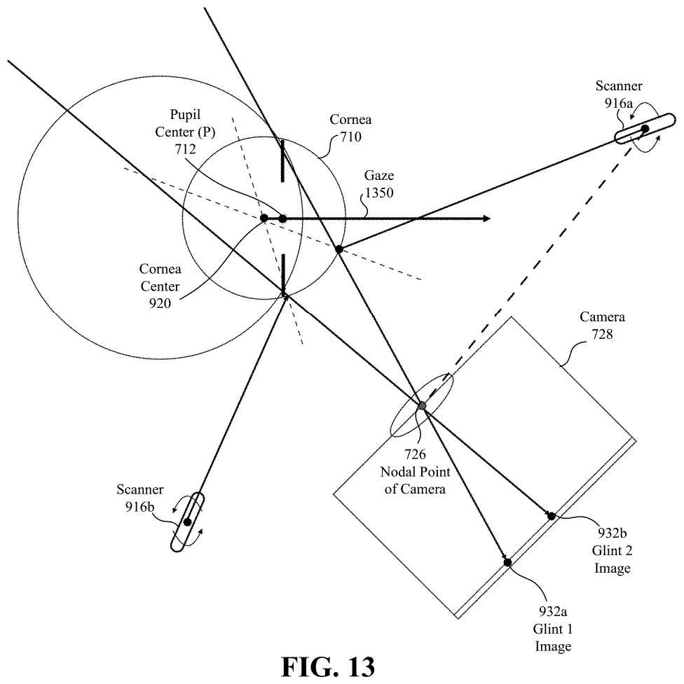

In some implementations, two scanners may be used to determine the cornea center and the gaze direction is determined using either the pupil center or the eyeball center. For example, FIG. 13 illustrates a functional block diagram illustrating gaze tracking using two variable angle light sources (e.g., a scanners 916a, 916b) at known positions. The cornea center 920 is determined based on glints from the light source directed by scanners 916a, 916b and the angles of the scanners 916a, 916b measured by the encoders. The pupil center 712 is also determined, for example, using image processing on the image data obtained by the camera 728. For example, based on determining the center of the iris. The gaze direction 1350 is determined as the vector from the cornea center 920 through the pupil center 712.

Numerous specific details are set forth herein to provide a thorough understanding of the claimed subject matter. However, those skilled in the art will understand that the claimed subject matter may be practiced without these specific details. In other instances, methods apparatuses, or systems that would be known by one of ordinary skill have not been described in detail so as not to obscure claimed subject matter.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, and/or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modification may be implemented by those skilled in the art without departing from the scope and spirit of the invention.

What is claimed is:

1. A method comprising:
at an electronic device having a processor:
producing a light beam via a light source, wherein the light beam is moved in multiple directions over time and a reflection from a portion of an eye is received at a sensor when the light beam is produced in a first direction of the multiple directions;
determining a second direction of the reflection based on data produced by the sensor;
determining a cornea center of the eye based on the first direction of the light beam and the second direction of the reflection determining a pupil center of the eye or an eyeball center of the eye; and
determining a gaze direction by determining a direction from the pupil center through the cornea center or a direction from the eyeball center through the cornea center.

2. The method of claim 1, wherein the cornea center is determined using a single glint.

3. The method of claim 1, wherein the gaze direction is determined by determining a direction from the pupil center through the cornea center.

4. The method of claim 3, wherein the pupil center is determined based on ambient light reflected from the eye and detected at the sensor or a second sensor.

5. The method of claim 3, wherein the pupil center is determined based on light from a diffuse light source detected at the sensor or a second sensor.

6. The method of claim 1, wherein the eyeball center is determined by fitting a sphere based on positions based on an eyeball position.

7. The method of claim 1, wherein the eyeball center is determined based on points on the cornea detected over time based on glints from light beams from the light source.

8. The method of claim 1, wherein the cornea position is determined based on determining a position of the glint and an average or previously measured cornea radius.

9. The method of claim 1, wherein the cornea center is determined based on a second reflection produced by a second light beam from a second light source, wherein the cornea center is determined by determining a position of the reflection on the cornea and a position of the second reflection on the cornea.

10. The method of claim 1 further comprising:
generating a three dimensional (3D) reconstruction of the cornea; and
determining the cornea center based on the 3D reconstruction.

11. The method of claim 1, wherein the light source is a laser, a vertical-cavity surface-emitting laser (VCSEL), a narrow beam light source, or a collimated light source.

12. The method of claim 1 further comprising moving the light beam over the eye via a scanner.

13. The method of claim 12 further comprising tracking a first angle of the light source while moving the light beam over the eye.

14. The method of claim 1 further comprising:
determining a first angle based on the first direction of the light source;
determining a second angle based on the second direction of the reflection; and
determining a position in three-dimensional (3D) space on the surface of the cornea based on the first angle and the second angle.

15. The method of claim 14 further comprising determining a center of the cornea based on the position on the surface of the cornea.

16. The method of claim 1, wherein the sensor is an event camera.

17. A device comprising:
a light source;
a scanner;
an encoder;
a pixel-based sensor;
a non-transitory computer-readable storage medium; and
one or more processors coupled to the non-transitory computer-readable storage medium, wherein the non-transitory computer-readable storage medium comprises program instructions that, when executed on the one or more processors, cause the system to perform operations comprising:
producing a light beam in multiple directions over time via the scanner;
encoding, via the coder, a first direction of the multiple directions when a reflection from a portion of an eye is received at the pixel-based sensor;
determining a second direction of the reflection based on pixel data produced by the sensor;
determining a cornea center of the eye based on the first direction of the light beam and the second direction of the reflection;
determining a pupil center of the eye or an eyeball center of the eye; and
determining a gaze direction by determining a direction from the pupil center through the cornea center or a direction from the eyeball center through the cornea center.

18. The system of claim 17, wherein the pixel-based sensor is an event camera.

19. The system of claim 17, wherein the light source is a laser, a vertical-cavity surface-emitting laser (VCSEL), a narrow beam light source, or a collimated light source.

20. A non-transitory computer-readable storage medium, storing program instructions computer-executable on a computer to perform operations comprising:
producing a light beam via a light source, wherein the light beam is moved in multiple directions over time and a reflection from a portion of an eye is received at a sensor when the light beam is produced in a first direction of the multiple directions;
determining a second direction of the reflection based on data produced by the sensor;
determining a cornea center of the eye based on the first direction of the light beam and the second direction of the reflection;
determining a pupil center of the eye or an eyeball center of the eye; and
determining a gaze direction by determining a direction from the pupil center through the cornea center or a direction from the eyeball center through the cornea center.
